(19) United States
(12) Patent Application Publication
(10) Pub. No.: US 2014/0165926 A1
(43) Pub. Date: Jun. 19, 2014

(54) DIAPER AND METHOD FOR ITS MANUFACTURE

(71) Applicant: PUDDLES BE GONE, Brenham, TX (US)

(72) Inventor: Diane Marks, Brenham, TX (US)

(73) Assignee: PUDDLES BE GONE, Brenham, TX (US)

(21) Appl. No.: 13/720,810

(22) Filed: Dec. 19, 2012

(51) Int. Cl. A61F 13/493; A01K 21/00; A61F 13/15

(52) U.S. Cl. CPC A61F 13/493; A61F 13/15747; A01K 21/00
USPC 119/838; 604/385.24; 493/393

(57) ABSTRACT

A diaper and method for its manufacture are disclosed. The diaper comprises one gathered lengthwise edge with an elastic material for that edge to expand and contract. The other lengthwise edge is static in length and longer than the gathered edge. The disparity in length tapers the diaper toward the gathered edge. The diaper is secured around a male quadruped with the gathered edge being around the quadruped's torso and the static edge fitting immediately anterior to its hind legs.

### BACKGROUND OF THE INVENTION

1. Field of the Invention

This invention relates to a diaper and more particularly relates to a diaper for a male quadruped that provides an easy to fit and form fitting solution for catching certain bodily excrement of the male quadruped.

2. Description of the Related Art

Diapers for a male quadruped have inconsistent fittings around the body of the recipient. Quadrupeds' varying shapes and sizes combined with their diverse walking styles and anatomical structures lead to the shortcoming of the prior art, improper fitting or painful rubbing of the diaper against any number of body parts of the quadruped.

From the foregoing discussion, it should be apparent that a need exists for a quadruped diaper that fits appropriately on a male quadruped and caters to the male quadruped's anatomical structure. Beneficially, such a diaper would account for the movement of different species of male quadrupeds and provide adequate areas with which to capture intended excrement from the male quadruped without discomfort.

### SUMMARY OF THE INVENTION

The present invention has been developed in response to the present state of the art, and in particular, in response to the problems and needs in the art that have not yet been fully solved by currently available diapers for male quadrupeds. Accordingly, the present invention has been developed to provide a diaper for a male quadruped and method for manufacturing a diaper for a male quadruped that overcome many or all of the above-discussed shortcomings in the art.

The diaper is provided with an exterior layer, interior layer, and absorbent layer that is between the exterior and interior layers. The exterior layer is liquid impermeable to prevent the excrement from a male quadruped from seeping through. The interior layer is liquid permeable to allow the excrement to seep through to the absorbent layer where it will be absorbed.

The diaper, in one embodiment, is configured at one lengthwise edge by gathering the material at the edge of the diaper such that the edge that is gathered becomes shorter in length than the opposite edge. The opposite edge may not be gathered, but in one embodiment, has a padded border which when fastened to a male quadruped creates a gap between the interior layer and the torso of the male quadruped.

The diaper is further configured, in one embodiment, with an elastic piece that is located near the gathered edge. The elastic piece may expand when acted on by a force and contract when the force is removed. This expansion and contraction of the elastic piece may also cause the material that has been gathered at one lengthwise edge to contemporaneously expand and contract.

In a further embodiment, the apparatus may be configured with one or more fasteners attached to a widthwise edge. The fasteners may extend longitudinally from that widthwise edge.

The diaper is further configured with one or more fastening areas to receive the one or more fasteners. The one or more fastening areas may be located on a widthwise edge opposite from the widthwise edge where the fasteners are located.

In one embodiment, the diaper is intended for the edge with the gathered material to secure around the torso of the male quadruped with the opposite edge to secure immediately anterior to the hind legs.

The diaper may have a barrier located near the edge with the gathered material that protrudes from the interior layer to block excretions from passing. In one embodiment, the barrier is liquid impermeable.

The diaper, in one embodiment, is configured to be tapered towards the edge with the gathered material. The material may be gathered such that when acted on by a force, that edge may extend to be longer in length than the opposite edge such that the diaper may taper towards the opposite edge.

The diaper may be configured to be discarded after one use.

A method of the present invention is also presented for manufacturing a diaper for a male quadruped. The method in the disclosed embodiments substantially includes the steps necessary to carry out manufacturing the diaper for a male quadruped. In one embodiment, the method includes attaching an exterior liquid impermeable layer to an interior liquid permeable layer with an absorbent layer between.

The method also may include gathering material of a first lengthwise edge of the attached layers to shorten that edge thus tapering the diaper toward that edge. In one embodiment, the method requires securing an elastic material to the attached layers near the first lengthwise edge. Securing the elastic material may allow the first lengthwise edge to expand and contract.

In one embodiment, a second lengthwise edge, which is opposite the first lengthwise edge, neither expands nor contracts. The method may include padding the first lengthwise edge with padding thick enough to establish a gap between the body of the male quadruped and the interior liquid permeable layer.

The method also may include affixing one or more fastening modules to one of the widthwise edges of the diaper, which one or more fastening modules extend out longitudinally from that edge. In that embodiment, the method includes affixing one or more fastening receptacles to the other widthwise edge, which one or more fastening receptacles are located wholly on the exterior layer and are configured to receive the fastening modules.

In a further embodiment, the method includes affixing a liquid impermeable barrier that is attached to the interior layer near the edge where the material was gathered and extends outward from the interior layer. The liquid impermeable barrier may expand and contract in response to expansion and contraction of the elastic material.

The method may include tapering the diaper toward the second lengthwise edge by expanding the first lengthwise edge. In one embodiment, the method includes padding the second lengthwise edge to both provide comfort for the male quadruped and form a spatial cavity with which to catch certain excrements.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a view of one embodiment of the inner surface of the diaper in accordance with the present invention;

FIG. 2 is a view of one embodiment of the exterior surface of the diaper in accordance with the present invention;

FIG. 3 is a view of a cross section of one embodiment of the diaper in accordance with the present invention;

FIG. 4 is a view of one embodiment of the diaper positioned around a male quadruped;

FIG. 5 is a view of one embodiment of the inner surface of the diaper in accordance with the present invention;

FIG. 6 is a view of a cross section of one embodiment of the diaper in accordance with the present invention;

FIG. 7 is a view of one embodiment of the diaper positioned around a male quadruped; and

FIG. 8 is a schematic flow chart diagram illustrating one embodiment of a method for manufacturing a diaper for a male quadruped in accordance with the present invention; and

FIG. 9 is a schematic flow chart diagram illustrating one embodiment of a method for manufacturing a diaper for a male quadruped in accordance with the present invention.

### DETAILED DESCRIPTION OF THE INVENTION

Many of the functional units described in this specification have been labeled as modules, in order to more particularly emphasize their implementation independence. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. One skilled in the relevant art will recognize that the invention may be practiced without one or more of the specific details, or with other methods, components, materials, and so forth. In other instances, well-known structures, materials, or operations are not shown or described in detail to avoid obscuring aspects of the invention.

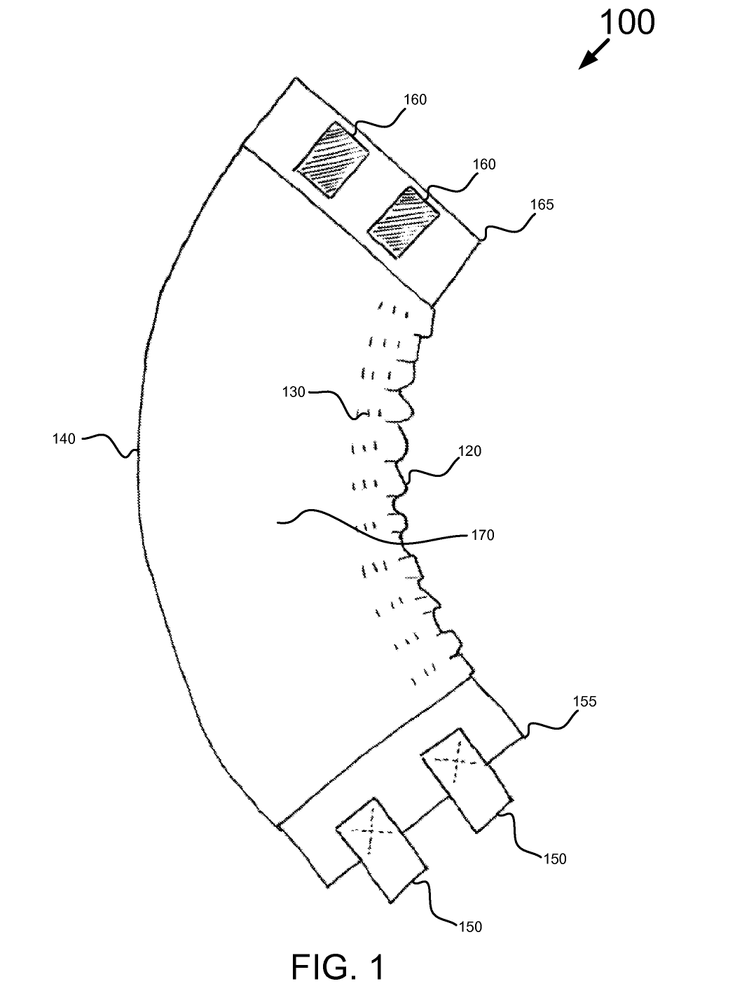

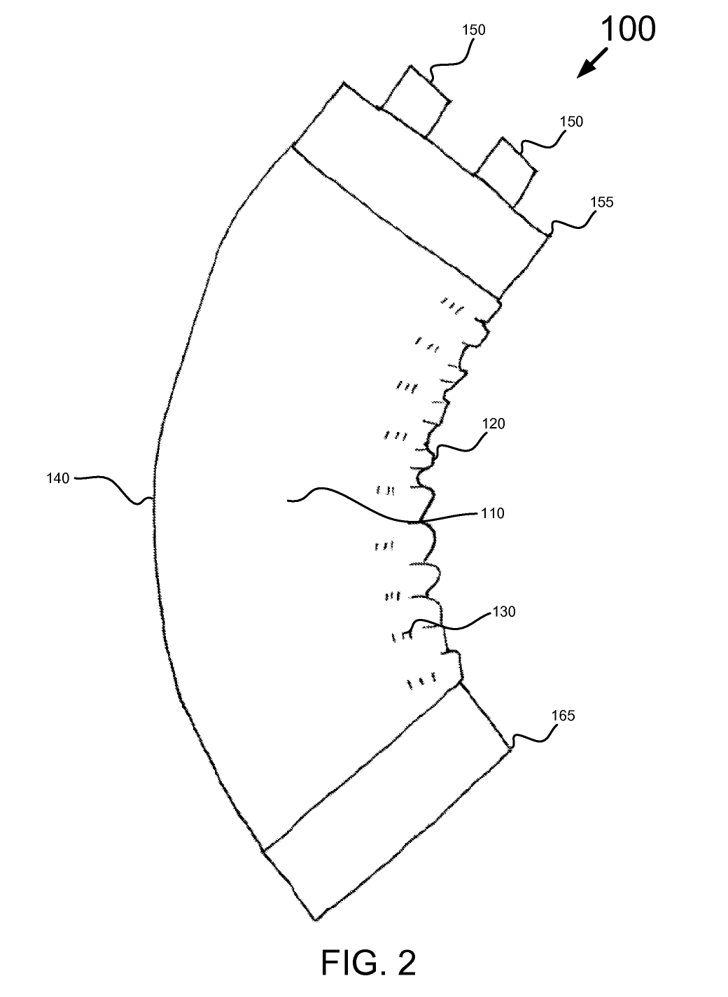

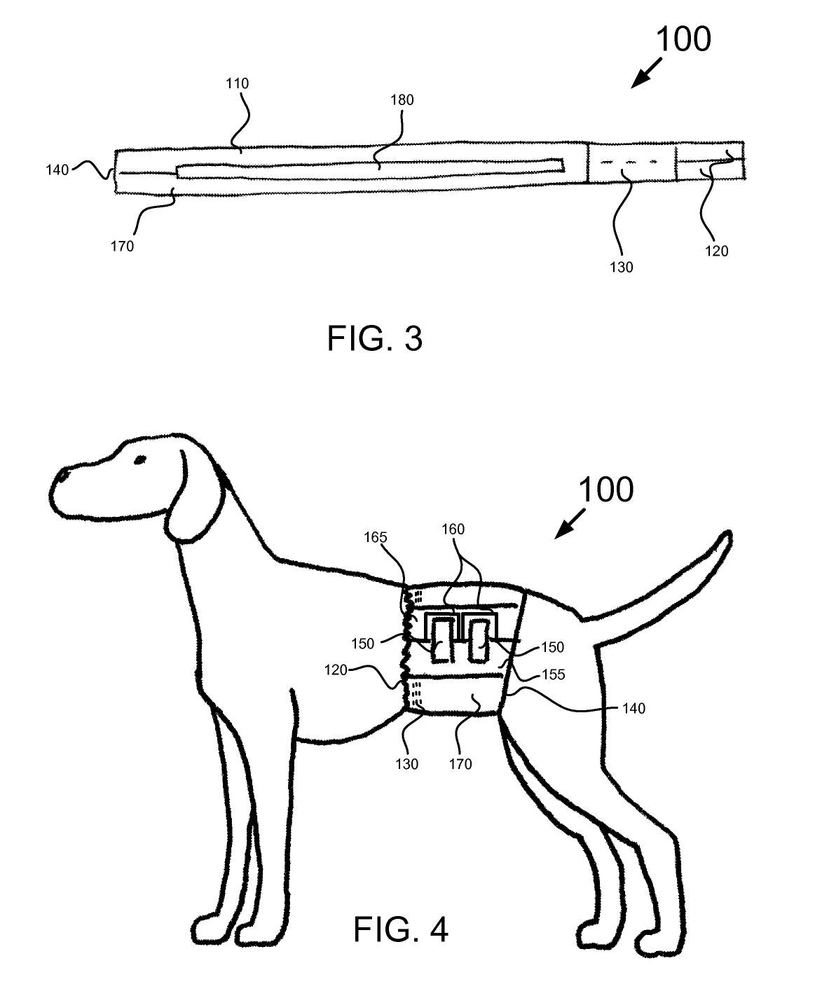

FIGS. 1 through 4 depict a diaper 100 in accordance with the present invention viewed from various vantage points. The depicted diaper 100 includes an interior layer 110, a gathered edge 120, an elastic module 130, a second lengthwise edge 140, one or more fastening modules 150, a first widthwise edge 155, one or more fastening receptacles 160, a second widthwise edge 165, an exterior layer 170, and an absorbent layer 180. The diaper 100 is one example of a diaper suitable of capturing certain excrement of a male quadruped and acting as a prophylactic in accordance with the present invention.

The interior layer 110 may be liquid permeable to allow liquid, such as certain excrement from a male quadruped, to permeate through the interior layer 110. In one embodiment, the interior layer 110 is connected to the exterior layer 170 with the absorbent layer 180 between the interior layer 110 and the exterior layer 170. The exterior layer 170 may be liquid impermeable to prevent liquid from seeping out of the diaper 100 or otherwise becoming perceptible. The interior layer 110 may be adjacent to the body of a male quadruped when the diaper 100 is secured around a male quadruped. In one embodiment, the absorbent layer 180 absorbs liquid, such as certain excrement from a male quadruped. The absorbent layer 180 may be made from any type of absorbing material or materials that may be held together as one conglomerate or loose but contained within the interior layer 110 and exterior layer 170.

In one embodiment, the gathered edge 120 is formed by gathering material of the interior layer 110 and exterior layer 170 at that edge of the diaper 100. Gathering the material to form the gathered edge 120 may allow the gathered edge 120 to be expanded and contracted. In one embodiment, the elastic module 130 is secured at a location near the gathered edge 120. The elastic module 130 may be secured directly adjacent to the gathered edge 120. The elastic module 130 may expand when a force is applied to it. The elastic module 130 may contract when a force is removed from it. In one embodiment, the gathered edge 120 expands and contracts in response to expansion and contraction of the elastic module 130.

In one embodiment, the second lengthwise edge 140 is not able to expand when a force is applied to it. The second lengthwise edge 140 may not be able to contract when a force is removed from it. In the depicted embodiment of the diaper 100, gathering material to form the gathered edge 120 causes the gathered edge 120 to be shorter than the second lengthwise edge 140, which tapers the diaper towards the gathered edge 120 when no force is applied to expand the elastic module 130. The tapered design may provide for the diaper 100 to fit securely and comfortably around a male quadruped as shown in FIG. 4.

In one embodiment, the one or more fastening modules extend out longitudinally from the first widthwise edge 155. The one or more fastening receptacles 160 may be wholly located inward from a second widthwise edge 165. The diaper 100 may be fastened securely around a male quadruped through the use of one or more fastening modules 150, in one embodiment, being attached to one or more fastening receptacles 160. The amount of one or more fastening modules 160 may not be the same as the amount of one or more fastening modules 150. In one embodiment, the one or more fastening modules 150 fasten to the one or more fastening receptacles 160 by use of a hook and loop system. The one or more fastening modules 150 may include a tacky substance which would stick to the one or more fastening receptacles 160, in which case the tacky substance could withstand being detached and reattached. The one or more fastening modules 150 may attach to the one or more fastening receptacles 160 by any means with which the two may be secured together so as to fasten the diaper 100 around a male quadruped.

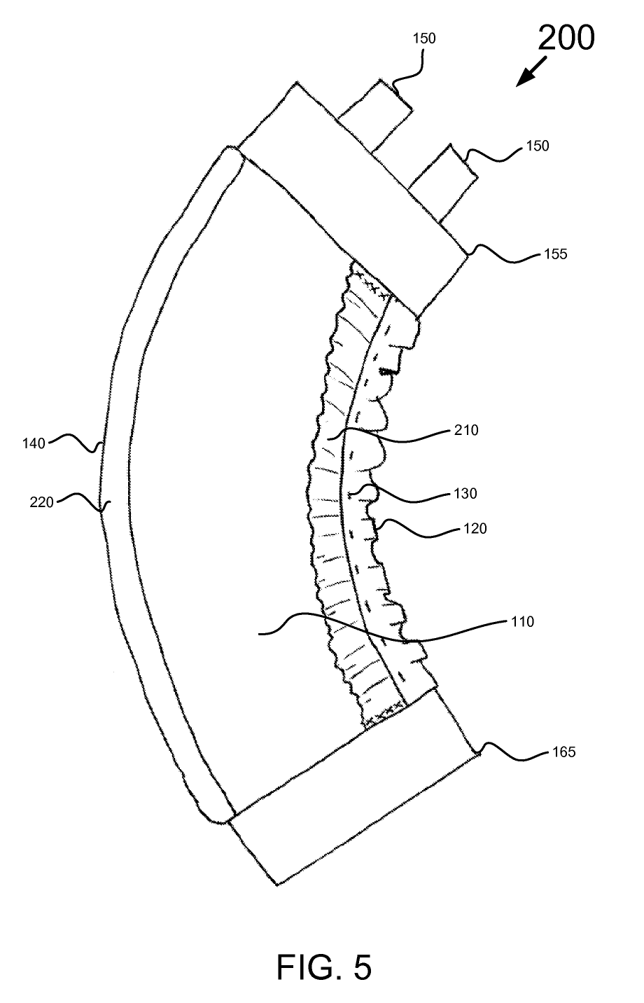

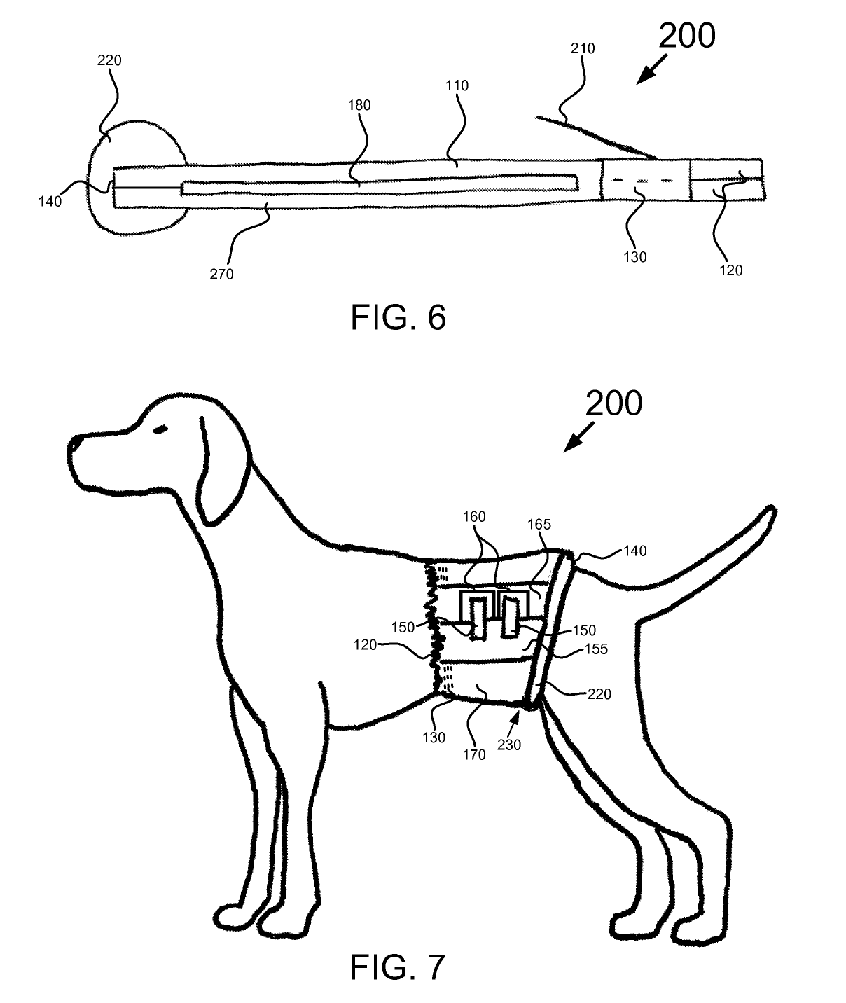

FIGS. 5 through 7 depict a diaper 200 in accordance with the present invention viewed from various vantage points. The depicted diaper 200 includes the interior layer 110, the gathered edge 120, the elastic module 130, the second lengthwise edge 140, the one or more fastening modules 150, the first widthwise edge 155, the one or more fastening receptacles 160, the second widthwise edge 165, the exterior layer 170, and the absorbent layer 180 of diaper 100. The diaper 200 also includes a barrier module 210, a padding 220, and a spatial cavity 230. The diaper 200 is one example of a diaper suitable of capturing certain excrement of a male quadruped and acting as a prophylactic in accordance with the present invention.

In one embodiment, the barrier module 210 is liquid impermeable to prevent excrement from a male quadruped from leaking, seeping, or otherwise not being contained within the diaper 200. The barrier module 210 may span the length of the diaper 200. The barrier module 210 may be shorter than the length of the diaper 200. In one embodiment, the barrier module 210 is attached near the gathered edge. The barrier module 210 may extend up from the inner layer 110 at a less than orthogonal or perpendicular angle toward the middle of the inner layer 110. The barrier module 210 may attach near the elastic module 130. The barrier module 210 may be able to expand when a force is applied to it and contract when the force is removed. In one embodiment, the barrier module 210 expands and contracts with the elastic module 130. When expanded, the angle from which the barrier module 210 extends from the inner layer 110 may increase.

The padding 220 may be affixed at the second lengthwise edge 140 of the diaper 200. The padding 220 is not able to expand when a force is applied to it. In one embodiment, the padding 220 provides comfort to a male quadruped. When the diaper 200 is secured around a male quadruped, as depicted in FIG. 7, the padding 220 comes in contact with the male quadruped, but the padding 220 is made with such a thickness that the portion of the inner layer 110 immediately adjacent to the padding 220 does not touch the body of the male quadruped, thus creating the spatial cavity 230.

The spatial cavity 230 may provide further comfort for the male quadruped. In one embodiment, the spatial cavity 230 facilitates receipt of excrement of a male quadruped. In one embodiment, the spatial cavity 230 acts as a collector of excrement of a male quadruped. With the excrement of the male quadruped collecting in the spatial cavity 230, the excrement is more likely to permeate through the inner layer 110 and be absorbed by the absorbent layer 180.

The schematic flow chart diagrams that follow are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

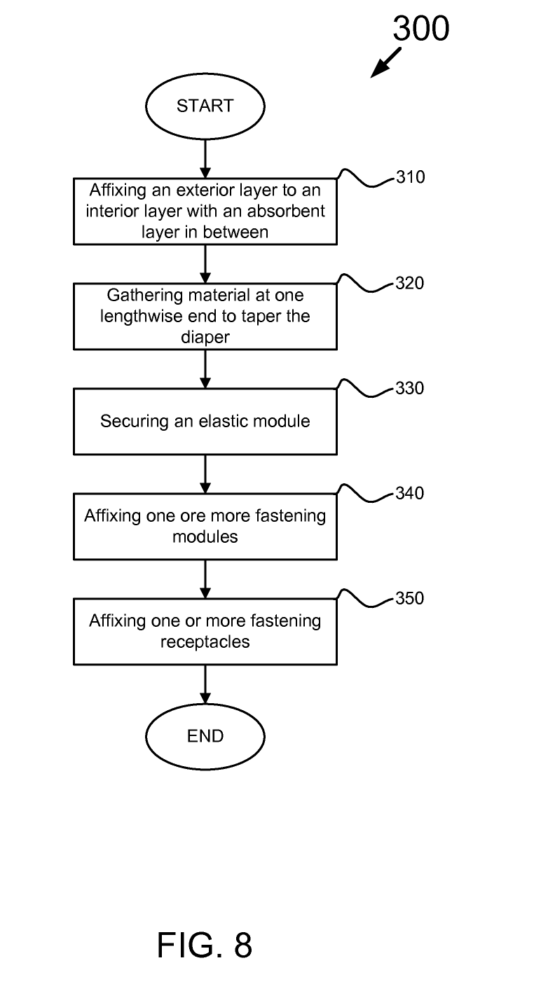

FIG. 8 is a schematic flow chart diagram illustrating one embodiment of a method for manufacturing a diaper for a male quadruped in accordance with the present invention. The method 300 includes a step 310 attaching an exterior layer that is liquid impermeable to an interior layer that is liquid permeable and encapsulating an absorbent layer between them, a step 320 gathering material at one lengthwise end, a step 330 securing an elastic material, a step 340 affixing one or more fastening modules, and a step 350 affixing one or more fastening receptacles. The depicted method 300 is used for producing a diaper for a male quadruped in accordance with the present invention.

In one embodiment, the step 310 includes a means for attaching a liquid impermeable layer to a liquid permeable layer. The absorbent layer may be placed between the exterior layer and interior layer prior to the two layers being attached to each other. Attaching the exterior layer to the interior layer may form a first and second widthwise end and a first and second lengthwise end. The step 310 may result in a diaper body.

In one embodiment, the step 330 includes gathering material from the exterior layer and the interior layer contemporaneously. The step 330 may include gathering equal amounts of material from the exterior layer and the interior layer. The step 330 may be at a first lengthwise end of the diaper body. In one embodiment, the step 330 includes gathering material in such a way that the gathered material may expand out to effectively be ungathered and contract back in to again be gathered.

The step 330 may include securing the elastic module to both the interior layer and the exterior layer. In one embodiment, the step 330 includes securing the elastic module near the gathered edge. The elastic material may expand when a force is applied to it and contract when the force is removed. The step 330 may include securing the elastic material to the diaper body such that the gathered edge expands and contracts in response to the elastic material expanding and contracting.

In one embodiment, the step 340 includes affixing one or more fastening modules to the first widthwise end of the diaper body such that the one or more fastening modules extend out longitudinally from the diaper body. The one or more fastening modules may be of a material designed to withstand multiple fastenings. The step 350 includes affixing one or more fastening receptacles to the second widthwise end such that the one or more fastening receptacles are wholly located inward from the second widthwise end and do not extend out from the diaper body. The one or more fastening receptacles may be of a material designed to allow fastening of the one or more fastening modules. The step 340 may not affix the same amount of fastening modules as the step 350 affixes fastening receptacles.

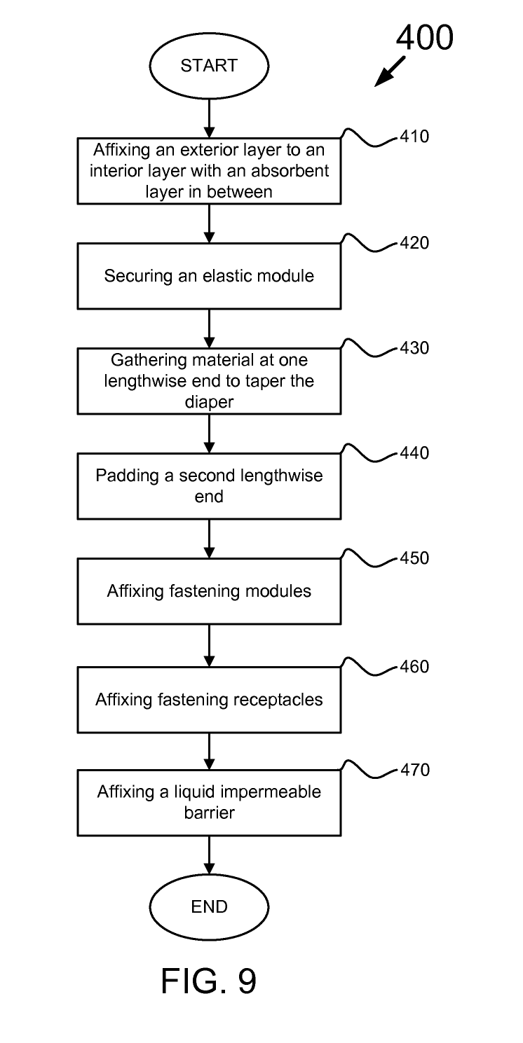

FIG. 9 is a schematic flow chart diagram illustrating one embodiment of a method for manufacturing a diaper for a male quadruped in accordance with the present invention. The method 400 includes the step 310 attaching an exterior layer that is liquid impermeable to an interior layer that is liquid permeable and encapsulating an absorbent layer between them, the step 320 gathering material at one lengthwise end, the step 330 securing an elastic material, the step 340 affixing one or more fastening modules, and the step 350 affixing one or more fastening receptacles as those steps are discussed in FIG. 8. The method 400 also includes a step 410 affixing a liquid impermeable barrier and a step 420 padding a second lengthwise end. The depicted method 400 is used for producing a diaper for a male quadruped in accordance with the present invention.

In one embodiment, the step 410 includes attaching the liquid impermeable barrier to the diaper body on the interior layer near the gathered edge. The barrier may extend at a less than perpendicular angle from the interior layer in a direction toward the middle of the interior layer. The barrier may be configured to expand when a force is applied to it and contract when the force is removed. In one embodiment, the step 410 includes affixing the barrier so that the barrier expands and contracts with the elastic material. The step 410 may include affixing the barrier so the angle at which the barrier extends from the interior layer increases as the barrier is expanded and decreases as the barrier is contracted.

The step 420 may include padding the second lengthwise end such that the amount of padding on the interior portion of the diaper is greater than the amount of padding on the exterior portion. The step 420 may include padding more on the interior layer than on the exterior layer. In one embodiment, the step 420 includes using a padding that neither expands nor contracts. The padding on the interior layer at the second lengthwise end may be designed to press against the male quadruped when the diaper is secured around the male quadruped. The padding may be of a thickness that forms a spatial cavity between the interior layer and the body of the male quadruped immediately proximal to the padding.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A diaper comprising:
an exterior layer, wherein the exterior layer is configured to be liquid impermeable;
an interior layer, wherein the interior layer is configured to be liquid permeable;
the exterior layer affixed to the interior layer to form a diaper body;
an absorbent layer enclosed between the exterior layer and the interior layer, wherein the absorbent layer is configured to absorb liquid;
a gathered edge, wherein the gathered edge is configured at a first lengthwise edge of the diaper;
a second lengthwise edge, wherein the second lengthwise edge is configured to be on the opposite end of the diaper body from the gathered edge;
the gathered edge further configured by gathering material of the diaper body together such that the gathered edge is shorter in length than the second lengthwise edge which tapers the diaper body in the direction of the gathered edge;
an elastic module, wherein the elastic module is configured to expand in response to a force and contract in response to removal of the force;
the elastic module further configured to be proximal to the gathered edge;
the gathered edge further configured to expand and contract in response to and contemporaneously with expansion and contraction of the elastic module;
the second lengthwise edge further configured to neither expand nor contract;
at least one fastening module affixed to and extending out longitudinally from the first widthwise edge;
a second widthwise edge, wherein the second widthwise edge is configured to be on the opposite end of the diaper body from the first widthwise edge;
at least one fastening receptacle affixed inward from the second widthwise edge configured to be wholly on the exterior layer; and
the at least one fastening module configured to attach to the at least one fastening receptacle to secure the diaper around a male quadruped such that the gathered edge is configured to secure around the torso of the male quadruped and the second lengthwise edge is configured to secure immediately anterior to the hind legs of the male quadruped.

2. The diaper of claim 1, further comprising padding, wherein the padding is affixed to the second lengthwise edge and is configured with a thickness to create a gap between the inner layer and the body of the male quadruped when the second lengthwise edge is secured around the male quadruped.

3. The diaper of claim 1 further comprising a barrier module affixed to the interior layer proximal to the elastic module, wherein the barrier module is configured to extend from the interior layer at a less than orthogonal angle toward the median of the interior layer.

4. The diaper of claim 3, wherein the barrier module is further configured to expand and contract in response to and contemporaneously with expansion and contraction of the elastic module.

5. The diaper of claim 3, wherein the barrier module is further configured to be liquid impermeable.

6. The diaper of claim 1, wherein the gathered edge is configured to expand to a length longer than the second lengthwise edge causing the diaper to taper toward the second lengthwise edge.

7. The diaper of claim 1, wherein the length of the elastic module is less than the length of the diaper.

8. The diaper of claim 1, wherein the length of the elastic module extends the length of the diaper.

9. The diaper of claim 1, wherein the diaper is configured to be discarded after use.

10. The diaper of claim 1, wherein the width of the absorbent layer is less than the width of the diaper body.

11. The diaper of claim 1, wherein the length of the absorbent layer is less than the length of the diaper body.

12. The diaper of claim 1, wherein the diaper is configured to be secured around a male quadruped such that the gathered edge is expanded around the male quadruped and subsequently allowed to contract securely around the torso of the male quadruped, the second lengthwise edge is wrapped anterior to the hind legs of the male quadruped, and the at least one fastening module is secured to the at least one fastening receptacle.

13. The diaper of claim 1, wherein the diaper is used as a prophylactic to prohibit the male quadruped from sexual interaction.

14. A method for manufacturing a diaper for a male quadruped, the method comprising:
affixing an exterior liquid impermeable layer to an interior liquid permeable layer with an absorbent layer therebetween to form a diaper body;
securing an elastic module near the first lengthwise end, wherein the elastic module is configured to expand in response to a force and contract in response to removal of the force;
gathering material at a first lengthwise end, wherein gathering material causes the diaper body to taper toward the first lengthwise end;
providing a second lengthwise end, wherein the second lengthwise end is configured to neither expand nor contract;
affixing fastening modules to a first widthwise end, wherein the fastening modules extend out longitudinally from the first widthwise end;
affixing fastening receptacles to a second widthwise end, wherein the fastening receptacles are affixed inward from the second widthwise end and configured to be wholly on the exterior layer;
the fastening receptacles configured to receive the fastening modules.

15. The method of claim 14 further comprising padding the second lengthwise end with a padding of a thickness to create a gap between the torso of the male quadruped and the interior liquid permeable layer.

16. The method of claim 14 further comprising affixing a liquid impermeable barrier module to the interior layer proximal to the elastic module, wherein the barrier module is configured to extend from the interior layer at a less than orthogonal angle toward the median of the interior layer.

17. The method of claim 16 further comprising configuring the liquid impermeable barrier module to expand and contract in response to and contemporaneously with expansion and contraction of the elastic material.

18. The method of claim 14 further comprising tapering the diaper body toward the second lengthwise end when the elastic material is expanded.

19. The method of claim 14 further comprising spanning the elastic material the entire length of the diaper body.

20. The method of claim 14 further comprising spanning the elastic material less than the entire length of the diaper body.
